(19) United States
(12) Patent Application Publication
(10) Pub. No.: US 2015/0196279 A1
(43) Pub. Date: Jul. 16, 2015

(54) SYNTHETIC-FOCUSING STRATEGIES FOR REAL-TIME ANNULAR-ARRAY IMAGING

(71) Applicants: Jeffrey A. Ketterling, New York, NY (US); Erwan Filoux, La Richie (FR)

(72) Inventors: Jeffrey A. Ketterling, New York, NY (US); Erwan Filoux, La Richie (FR)

(73) Assignee: RIVERSIDE RESEARCH INSTITUTE, New York, NY (US)

(21) Appl. No.: 14/659,857

(22) Filed: Mar. 17, 2015

Related U.S. Application Data
(63) Continuation-in-part of application No. 13/655,086, filed on Oct. 18, 2012.
(60) Provisional application No. 61/548,385, filed on Oct. 18, 2011.

(51) Int. Cl. A61B 8/08; A61B 8/00; A61B 8/14

(52) U.S. Cl. CPC A61B 8/5207; A61B 8/145; A61B 8/4494; A61B 8/5269

(57) ABSTRACT

A method to increase the image formation speed in an annular-array digital ultrasound system consisting of N elements with a plurality of transmit and receive channels. Selectively dropping one or more transmit or receive channels during image formation reduces the amount of data needed to form an image, thus increasing the image formation frame rate through faster scan speeds or reducing the amount of digitized data required to process an image. The improved frame rate does result in some reduction in resolution, SNR and potentially DOF, but these drawbacks can be mitigated by synthetically recovering missing transmit-to-receive data pairs.

### PRIORITY AND RELATED APPLICATIONS

This application is a Continuation-in-Part of U.S. patent application Ser. No. 13/655,086, filed Oct. 18, 2012, entitled “SYNTHETIC-FOCUSING STRATEGIES FOR REAL-TIME ANNULAR-ARRAY IMAGING” which claims priority to U.S. Provisional Patent Application Ser. No. 61/548,385, filed Oct. 18, 2011, entitled “METHOD FOR IMPLEMENTING SYNTHETIC FOCUSING WITH ULTRASOUND ARRAYS,” both of which are hereby incorporated by reference in their entirety. This invention was supported by a grant from the National Institutes of Health (EB008606).

### FIELD OF THE INVENTION

The present invention relates to methods of acquiring and forming images using an ultrasound annular array and, in particular, to a synthetic-focusing strategy to achieve enhanced frame rates or reduced image processing time by selectively removing an element from transmit or receive, but not both simultaneously, or using a single transmit element and one or more receive elements.

### BACKGROUND OF THE INVENTION

Annular arrays have a history that stretches back to the early days of array-based ultrasound imaging. The appeal of annular arrays is that with a limited number of elements they can provide a greatly improved depth of field (DOF) and improved lateral resolution over the DOF when compared with a single-element focused transducer with the same total aperture and focal length. The reduced channel count of annular arrays is attractive for reducing system complexity.

The main drawback of annular arrays is that they must be mechanically scanned to form a B-mode image. For low-megahertz systems, this was a major drawback because of relatively long lateral displacements and the difficulty in obtaining real-time frame rates. However, high-frequency ultrasound (HFU, >15-MHz) applications for which penetration depths are typically on the order of 1 to 3 cm and image widths are 1 to 2 cm, such as small-animal and ophthalmic imaging, are well suited for annular arrays.

The normal approach to imaging with annular arrays, just as in most modern array-based imaging systems, is to use a fixed number of transmit focal zones and then dynamically receive the return echoes to create the displayed B-mode image. The more transmit focal zones that are used, the better the image quality, but at a cost of reduced frame rate. An alternate imaging approach used with linear and phased arrays is to construct images from individual transmit-to-receive (TR) subapertures; this method of beamforming is generally referred to as synthetic-aperture (SA) imaging.

SA approaches originated with radar-based systems for which an airborne source and receiver were towed over a target, effectively creating a long antenna through a series of TR events. Ultrasound-based SA imaging differs from the radar version in that an ultrasound array has a fixed number of elements with known spacing, which means that the full aperture is already present and defined. The aperture is therefore not truly synthetic, but rather data collected from individual TR subapertures can be processed with appropriate delay-and-sum beamforming to synthetically focus to any position within the field of view. Unlike a typical radar-based system with a single TR pair, an ultrasound array can transmit using an arbitrary subaperture and the return echoes can be collected simultaneously on all of the elements of the array aperture or a subaperture. The implication is that collecting all individual TR element combinations from an ultrasound array permits synthetic focusing to any point in space and is mathematically equivalent to exciting all array elements simultaneously to focus to the same point on transmit and receive. Once data are collected, an image can be reconstructed with an arbitrary number of transmit focal zones and dynamic receive zones.

In the context of annular arrays and, in particular, spherically-focused annular arrays, it is possible to apply SA-imaging approaches, but the present invention is directed to a synthetic-focusing approach to take advantage of fundamental differences in how annular arrays operate when compared with linear and phased arrays. Linear and phased arrays are composed of elements with the same geometric properties, length scales on the order of a wavelength, and uniform spacing between elements. The elements have uniform, essentially omnidirectional, acoustic field properties and they can be interchanged, in the sense that a mechanical shift of the array is identical to an electronic shift of elements. The omnidirectional nature of the acoustic field allows focusing to any point within the 2-D field of view of the array.

In contrast, focused annular arrays have elements of non-uniform dimensions, typically with length scales greater than a wavelength. Each element has different acoustic field properties and the elements cannot be interchanged. In addition, the acoustic field is highly directional because of the large aperture and spherical curvature, and focusing can only be achieved in 1-D along the acoustic axis. The 1-D focusing nature of the beamforming is analogous to a dynamic change of the geometric focus, which is why the term synthetic focusing (SF) best describes the process.

One disadvantage of SA and SF imaging is that only part of the aperture is typically used on transmit, reducing the overall SNR and penetration depth. This is less of an issue with an annular array because of the geometrically focused field and the relatively large aperture. SA techniques also generally require multiple transmit events, which can reduce the overall frame rate and make the approach more susceptible to motion artifacts. With a five-element annular array, as described herein, motion artifacts are a minor concern because only five excitations are needed to capture all TR element pair data and this can be accomplished within a few milliseconds. Real-time frame rate is a bigger issue, particularly in a purely digital system because more transmit events mean more acquired data and more processing time required to form an image. It is therefore useful to explore SF methods that reduce the quantity of acquired data to reduce image processing time or reduce transmit events to permit higher imaging frame rates.

### BRIEF SUMMARY OF THE INVENTION

It has been previously demonstrated that SF with five-element annular arrays that use all 25 data TR combinations acquired over five passes were very effective at forming high-quality images when operating around 20 and 40 MHz. However, this approach only achieved frame rates on the order of 1 frame per second (fps), which was not sufficient for real-time applications such as ophthalmic imaging. In accordance with the present invention, a one-pass approach is examined using a five-channel pulser combined with SF strategies that remove channels from either transmit or receive, but not both simultaneously. Numerical simulations are performed to quantify the acoustic-field properties of each SF approach to understand how resolution, DOF, and SNR are affected. The SF approaches are then applied to data sets acquired from a wire phantom, an anechoic-sphere phantom, and in vivo mouse embryos. The invention focuses on five-ring arrays that operate at 18 and 38 MHz, but the general trends apply to any annular array.

### BRIEF DESCRIPTION OF THE DRAWINGS

FIG. 1 shows tables representing transmit-to-receive combinations for a five-ring annular array,

FIG. 2 shows an example of simulated data in accordance with the invention,

FIG. 3 shows a simulated synthetic-focusing strategy in accordance with the invention,

FIG. 4 shows a simulated depth of field example,

FIG. 5 shows a simulated and experimental SNR with a 38-MHz array,

FIG. 6 shows images of anechoic spheres obtained in accordance with the invention,

FIG. 7 shows a contrast-to-noise ratio for a 530-mm anechoic sphere, and

FIG. 8 shows a B-mode image using a 38-MHz annular array

### DETAILED DESCRIPTION OF THE INVENTION

In one embodiment of the present invention, 18 and 38 MHz annular arrays were used. These arrays were fabricated as described in IEEE Transactions on Ultrasonics, Ferroelectrics and Frequency Control, Vol. 52, No. 4, pp. 672-681, 2005, which is incorporated herein by reference. Each array consisted of five equal-area elements and the active acoustic component was either a polyvinylidene fluoride (PVDF) or poly(vinylidene fluoride-tetrafluoroethylene) (P(VDFTrFE)) film of 9 pm (38 MHz) or 25 pm (18 MHz) thickness. The 18-MHz arrays had a total aperture of 10 mm and a focal length of 31 mm. The 38-MHz arrays had a total aperture of 6 mm and a focal length of 12 mm. The acoustic field of each TR pair was calculated using a spatial impulse response (SIR) model. The SIR, h(r, t), of each element was calculated at a point r in space and then the waveform proportional to the TR receive voltage, E(r, t), was obtained from the convolution a′(t)*h_{T}*h_{R} where a′(t) is the derivative of the transducer surface velocity, h_{T} is the transmit SIR and h_{R} is the receive SIR. This expression was used to compute individual RF scan lines for every TR combination at a series of depths and lateral positions. The results represented the pulse/echo response from a point target, and moving the point target axially or laterally allowed for the calculation of DOF or the lateral point spread function. Gaussian white noise was added to the simulated RF data such that the SNR was at least 45 dB relative to the magnitude of the TR signal at the geometric focus. The simulated SNR values were selected to follow experimental conditions. SNR was defined as the ratio of the peak signal value to the root mean square (RMS) background noise.

The RF data simulations were performed using an 18- or 38-MHz, 3-cycle sinusoid weighted with a Hamming window. The 18-MHz simulations used a 100 ps time step, 41 focal zones spanning from 16 to 46 mm in 1 mm steps, and a 0.8 mm lateral span with 40-μm spacing. The 38-MHz simulations used a 50 ps time step, 41 focal zones spanning from 6 to 18 mm in 1 mm steps, and a 0.4 mm lateral span with 20-pm spacing. Before storing the simulated data, the 18-MHz array data were resampled to 250 MHz and the 38-MHz array data to 1 GHz. These data were used to simulate the effect of beamforming approaches on SNR, lateral resolution, and DOF.

SF of digitized RF data was accomplished by applying an appropriate round-trip delay to each TR pair for a given focal depth and then summing the data to create a locally focused region. This process can be repeated to create an arbitrary number of focal zones. To focus the array at a depth f, the one-way delay tₙ of each element is

$${t}_{n} = \frac{{a}_{n}^{2} ( 1 / R - 1 / f )}{2 c}$$

where R is the geometric focal length, c is the speed of sound, and aₙ is the root mean square of the inner and outer radius of the nth array element. The round-trip delay for focusing at a depth f is found from the sum of the TR delays t^{tot}=t^{T}+t^{R}. To simulate a single-element transducer that has the same total aperture and geometric focus as the annular array, the RF data from the TR data pairs are simply summed with t^{tot}=0.

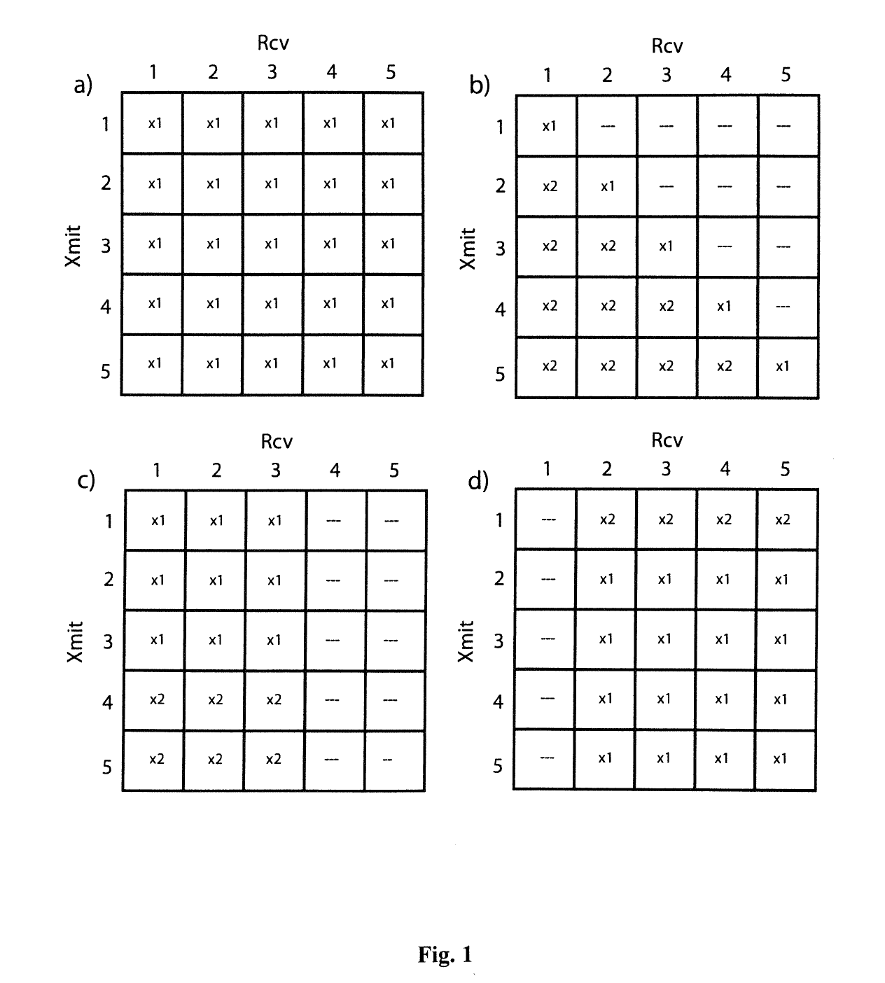

The initial approach to beamforming made use of all 25 individual TR pairs because images were post-processed and processing time and frame rate were not a major concern; see FIG. 1(a). However, real-time imaging requires frame rates of at least 6 fps in order to allow the image on the screen to present minimal lag-time time when an ultrasound probe is moved. For an annular array, the mechanical scan speed and image processing time are two items that can be optimized for real-time imaging. For a fast scanning motor, the number of transmit events represent a limiting factor in ultimate frame rate. For processing time, the amount of acquired data impacts the data transfer time and computational time to form an image.

One simple method to reduce acquired data or to reduce the number of transmit events is to take advantage of the TR equivalency of rings 1-to-5 and 5-to-1. This equivalency means that if one TR signal is eliminated and the remaining TR signal is doubled, the acoustic beam properties will remain unchanged. Using this approach, the 25 TR pairs from a five-ring annular array can be reduced to 15 unique TR pairs, of which 10 have a reciprocal TR pair; see FIG. 1(b). The symmetry can be exploited by eliminating one or more channels on either transmit or receive and then recovering the missing TR pairs by doubling whatever reciprocal TR pairs were acquired; see FIG. 1(c). With this technique, the full aperture is used either on transmit or on receive and the doubling of missing TR pairs minimizes the loss of resolution, SNR and DOF amplitude relative to the case using the full aperture on transmit and receive. This approach has the benefit of simplifying system complexity by reducing the number of pulser channels or digitization channels.

The FIG. 1 tables represent all TR combinations for a five-ring annular array. An x1 indicates data are used without modification, x2 indicates a doubling of magnitude, and—means no data are acquired. (a) Full SF with all 25 TR pairs is done without any adjustments. Case (a) is mathematically equivalent to case (b), in which one component of a reciprocal pair is removed and the partner is doubled. (c) If receive channels 4 and 5 are removed, doubling the appropriate reciprocal pairs recovers 6 of the 10 lost TR pairs. (d) If the central element, channel 1, is removed on receive, five TR pairs are lost but four of them can be recovered.

It is possible to examine the SF beamforming strategy of removing receive channels and doubling the amplitude of TR pairs that are the reciprocal of the dropped pairs. Beamforming is first applied to the simulated RF data to quantify changes in lateral beamwidth, DOF, and SNR. The case of all 25 TR data sets forms the gold standard for optimal beam characteristics. These SF strategies highlight the effect on the beam properties when the outermost one, two, three, or four annuli are removed from SF on receive.

In vitro and in vivo data were collected with the annular arrays to observe the SF strategies in practice and to benchmark their effect on system frame rate. The basic experimental system has been described previously in IEEE Transactions on Ultrasonics, Ferroelectrics and Frequency Control, Vol. 53, No. 3, pp. 623-630, 2006, which is hereby incorporated herein by reference, but various modifications have been made to improve system speed. The experimental system consisted of motion, digitization, and pulsing subsystems that were integrated into a PXI-based chassis (PXI-1042Q, National Instruments Corp. [NI], Austin, Tex.) under PC control (2.93-GHz Core i3, Intel Corp., Santa Clara, Calif.). The motion subsystem was composed of a motion-control card (PXI-7354, NI) and a high-speed linear actuator (LA535, SMAC Inc., Carlsbad, Calif.) with 23 mm of total travel. The digitizer subsystem consisted of three 2-channel, 8-bit digitizers (PXI-5154, NI). The pulsing subsystem was composed of a monocycle pulser (Panametrics 5900, Olympus NDT, Waltham, Mass.) and, on the receive side, 46-dB preamplifiers (AU-1313, MITEQ, Inc., Hauppauge, N.Y.). Because only a single pulsing channel was available, a multiplexer (Model 40-834, Pickering Interfaces Inc., Portland, Oreg.) was used to select the excitation element and five passes across the scan object were required to obtain all 25 TR pairs. The overall system was fully automated using a LabVIEW-based (NI) software tool.

For real-time frame-rate benchmarks, a one-pass approach was adopted by triggering the digitizers as if a five-channel pulser was being used in the system and acquiring noise data on the digitizers. The real-time benchmarks represented the time to translate the motor, digitize and transfer data, subtract the mean from each RF line, perform SF, log compress the data using a lookup table, and display the final image. A counter/timer card (PXI-6602, NI) was used to generate five staggered triggers that triggered a virtual five-channel pulser. These triggers were routed through an OR gate (M74HC4078, STMicroelectronics N.V., Geneva, Switzerland) and the OR gate output was used to trigger the digitizers. The sequence of triggers was generated once per spatial location and the trigger delay between channels was selected to avoid interference from the previous transmit event.

A wire phantom with a single, 15-pm-diameter wire was utilized to obtain the 25 TR combinations for the 18- and 38-MHz annular arrays at 1 mm axial intervals. Data were acquired from 8 to 20 mm at 50-pm lateral spacing for the 38-MHz array and from 18 to 50 mm at 100-pm spacing for the 18-MHz array. For both arrays, a 250-MHz sampling rate was used. The data were then processed using the various SF strategies and lateral beamwidth, DOF, and SNR were calculated.

In vitro data from each TR pair of the 18- and 38-MHz annular arrays were acquired from an anechoic phantom that was specifically designed for HFU use. The phantom consisted of eight sections containing a background material with a uniform distribution of 6.5-pm glass beads along with anechoic spheres of uniform, decreasing size (1090, 825, 530, 400, 300, 200, 137, and 100 pm) and a ninth slab devoid of spheres that contained only the background material. The spheres and the background were made from a mixture of preservative, agarose, and bovine milk. The attenuation coefficient at 40 MHz was ≈0.61 dB/cm/MHz in the background material and 0.58 dB/cm/MHz in the spheres. The speed of sound was ≈14540 m/s. The SF strategies were applied to the phantom data to quantify the contrast-to-noise ratio (CNR) along with the minimum sphere diameter that could be resolved. Data were acquired at a 250-MHz sampling rate with a 50-μm lateral spacing for the 38-MHz array and a 100-μm spacing for the 18-MHz array.

The imaging performance in terms of CNR of the different SF strategies was evaluated using spheres that were easy to detect using the techniques described in IEEE Transactions on Ultrasonics, Ferroelectrics and Frequency Control, Vol. 58, No. 5, pp. 994-1005, May, 2011. For the 38-MHz array, the 530-μm spheres were used, and for the 18-MHz array, the 1090-μm spheres were used. In addition, smaller spheres were imaged to show the evolution of detection capability as outer channels were dropped during SF. The spheres at the detection limit of the system were 200 μm for the 38-MHz array and 400 μm for the 18-MHz array. Detection of spheres was implemented with a semi-automated approach using MATLAB (The MathWorks Inc., Natick, Mass.). After calculating the envelope and log compressing the RF data, noise was reduced using a median filter and the image was smoothed using a Gaussian low-pass filter. Spheres were then detected using a simple threshold while taking into account the depth-based attenuation within the phantom. Then, square regions-of-interest (ROIs) the size of the theoretical radius of the spheres were defined around their detected center and similar ROIs were defined in the background at the same depths as the spheres. The statistical properties of the mean value, μ, and standard deviation, σ, of the envelope-detected RF signals inside these ROIs were measured. The CNR of the spheres was then calculated using the relation

$$CNR = \frac{\vert {μ}_{B} - {μ}_{S} \vert}{\sqrt{{σ}_{B}^{2} + {σ}_{S}^{2}}} ,$$

where μ_{B} and σ_{B} are the characteristics of the background and μ_{S} and σ_{S} are those of the anechoic spheres.

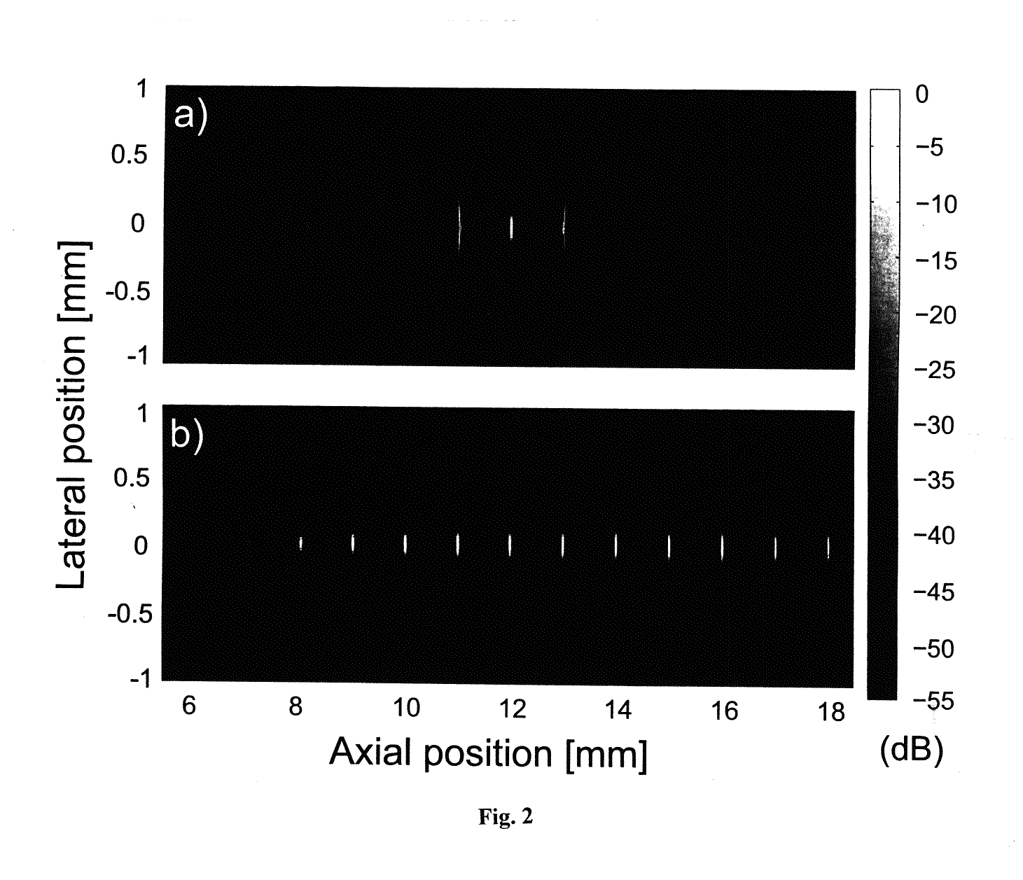

An example of simulated TR data for the 38-MHz annular array is shown in FIG. 2. The simulation represents the point-spread function at 1-mm axial intervals centered around the 12-mm geometric focus and can be interpreted as a B-mode image of wires at a series of depths. FIG. 2(a) represents the fixed-focus case with no delays applied to the TR data and is equivalent to a single-element transducer with a 12-mm geometric focus and 6-mm aperture diameter. The acoustic field shows the characteristic minimum lateral beamwidth and maximum amplitude at the geometric focus, and then the beamwidth and amplitude degrade when moving away from the focus. In contrast, full SF of all 25 TR pairs (FIG. 2(b)) revealed a slowly increasing beamwidth starting near 8 mm and less variation in the peak amplitudes when moving away from the geometric focus. At 12 mm, the SF case is the same as the fixed-focus case because no delays were applied.

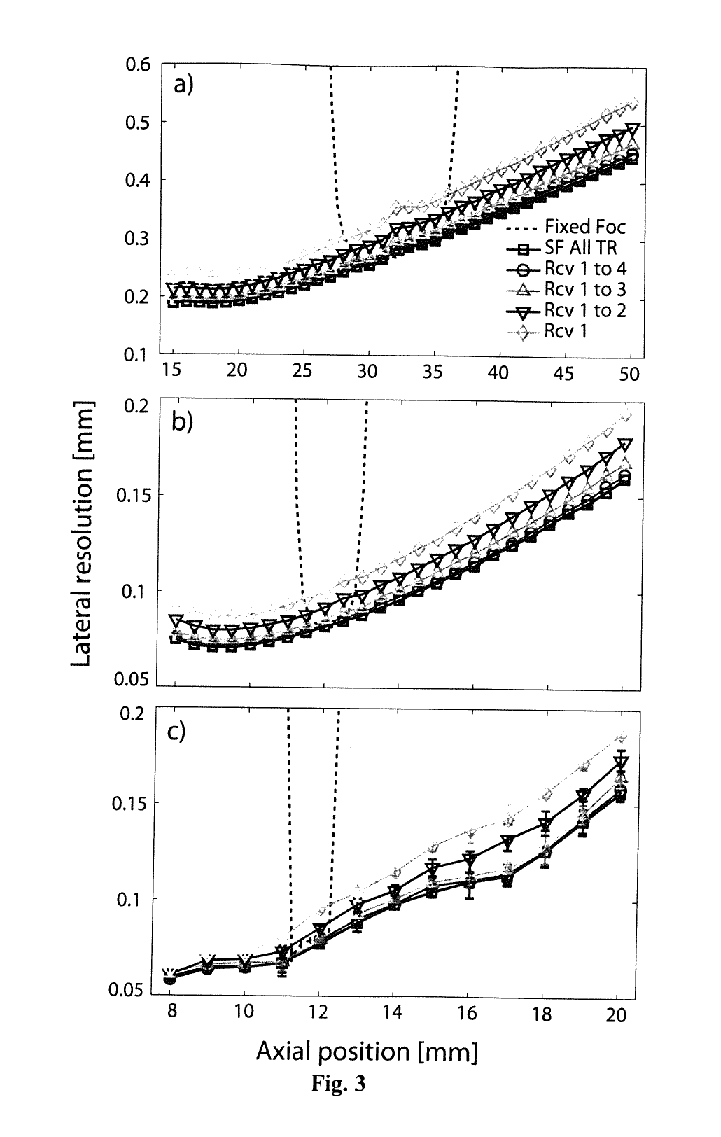

The −6-dB lateral beamwidths of the 38—(FIG. 3(a)) and 18-MHz (FIG. 3(b)) annular arrays were calculated at a series of 1-mm axial intervals for the SF strategies of fixed focusing with no delays applied, full SF with all 25 TR pairs, and transmitting on all five elements with the outermost one, two (FIG. 1(c)), three, or four receive annuli removed. Compared with fixed focusing, all the SF methods showed a dramatic improvement in lateral resolution outside the region of the geometric focus. At the geometric focus, the SF case with all 25 TR pairs and the fixed-focus case overlap, as would be expected.

FIG. 3 shows simulated −6-dB lateral beamwidth versus axial distance for fixed-focusing and SF strategies using the (a) 18- and (b) 38-MHz arrays along with (c) experimental wire phantom results using the 38-MHz array. Measurements were performed five times for each axial position of the wire target and the error bars represent the maximum and minimum amplitudes of the measurements. The full set of 25 TR pairs had the smallest beamwidths over the axial range and the beamwidth incrementally increased as the outer receive elements were removed.

For both array geometries, as the outer receive channels were removed one by one, the lateral resolution degraded. For the case with element 5 removed on receive, 24 effective TR pairs were used (20 unique TR pairs with four reciprocal TR pairs). As additional outer receive channels were removed, the number of effective TR pairs used for SF falls to 21, 16, and, finally, to 9 for the SF case with only the central channel, element 1, receiving. Using the full 25-TR case as the reference, removing the outermost receive element degraded lateral resolution by 1.4%, the outer two by 5.5%, the outer three by 12.2%, and the outer four by 22%. The degradation in resolution would have been somewhat higher without the strategy of doubling TR data to account for missing TR pairs.

Experimental wire phantom results using the 38-MHz array (FIG. 3(c)) show similar trends to the simulations. Some small differences can be observed, most likely caused by imperfections in the array geometry during fabrication and variations in the sensitivities of the elements. The experimental results for the 18-MHz array showed similar agreement to the theoretical predictions.

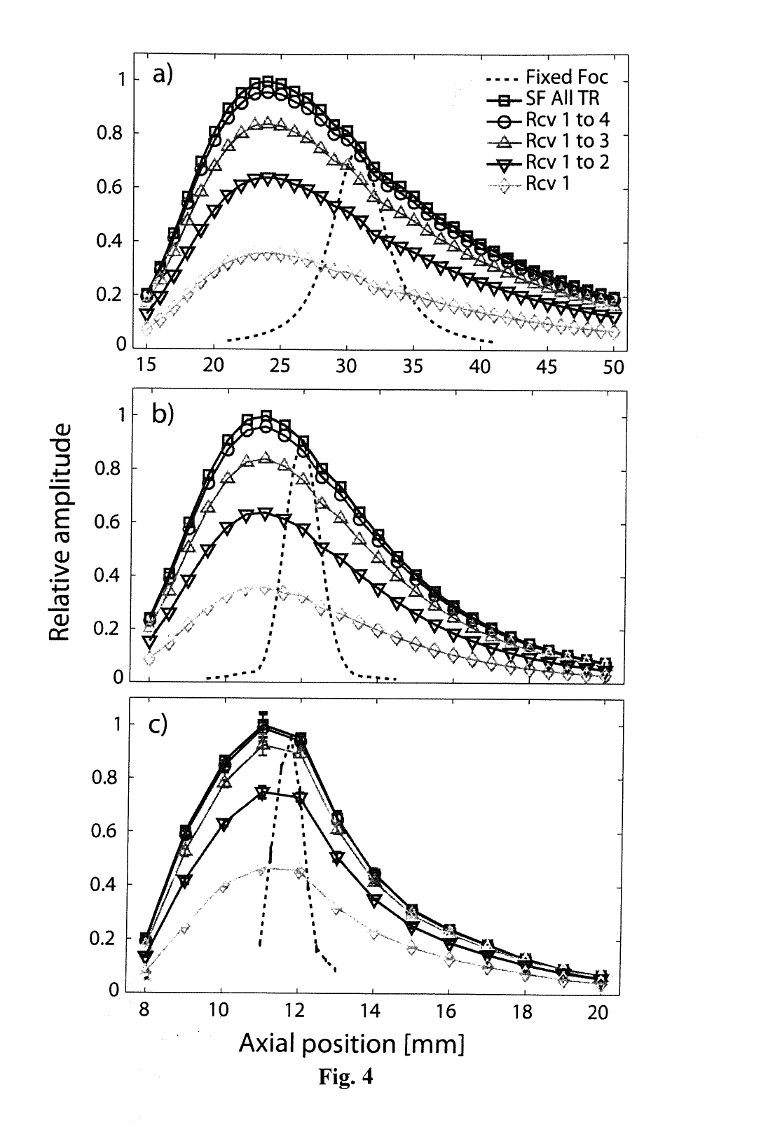

FIG. 4. shows simulated DOF for fixed-focusing and SF strategies using the (a) 18- and (b) 38-MHz arrays along with (c) experimental wire phantom results using the 38-MHz array. Measurements were performed five times for each axial position of the wire target and the error bars represent the maximum and minimum amplitudes of the measurements. All curves were normalized to the peak amplitude using the full set of 25 TR pairs. Removing the outer elements on receive reduced the amplitude of the DOF but the full-width at half-maximum (FWHM) values stayed the same for each curve.

The effects of the SF strategies on DOF for the 18- and 38-MHz arrays are observed in FIG. 4 and the improvements in DOF versus a fixed-focus transducer are evident. Using the full 25-TR case as the reference curve, it can be seen that removing the outer receive channels progressively lowers the overall amplitude profile of the DOF. This result was expected because the SF process is a summation of RF data and removing TR pairs lowers the maximum value that can be obtained for the total amplitude. The 18- and 38-MHz curves decreased by the same scaling factor when the outer receive channels were removed one by one. With the full 25-TR case as the reference, removing the outermost receive channel decreased the DOF magnitude by 4%. The removal of the remaining elements, one by one, decreased the amplitude by a further 16, 36, and 64%. The experimental DOF results for the 38-MHz array (FIG. 4(c)) showed similar trends to the simulations, but the overall decrease in DOF magnitude was slightly lower, partly because of the lower sensitivity of the outer elements of the array. The experimental results for the 18-MHz array showed similar agreement to the predictions.

When the various simulated DOF cases were normalized to one, the curves completely overlapped and the FWHM values were identical for all SF approaches (19 mm for the 18-MHz array and 5.7 mm for the 38-MHz array). This behavior is to be expected because of the relation between DOF and f-number (DOF ∝ f-number²; f-number=focal length/diameter). The central element has the smallest effective diameter and, thus, the largest f-number of all of the array elements. Therefore, when the central element is active, it dominates the DOF. In terms of imaging, this implies that removing the outer elements has no impact on overall DOF but, as described previously, the lateral resolution and overall signal magnitude will be degraded.

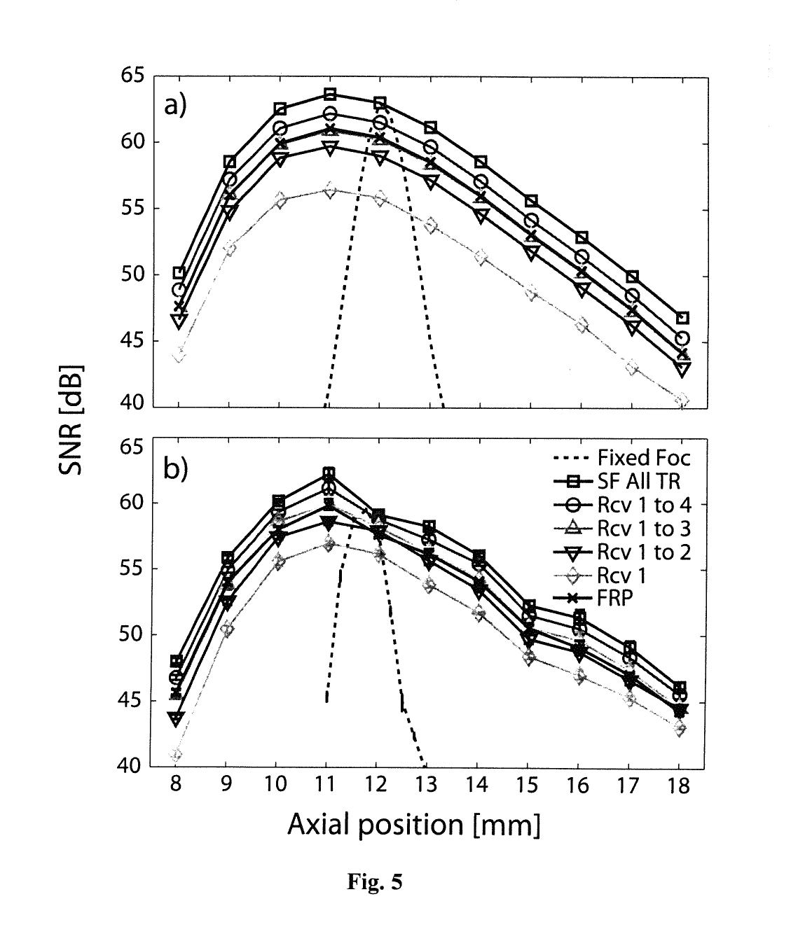

FIG. 5. shows (a) simulated and (b) experimental SNR with the 38-MHz array for fixed focusing, full 25 TR pairs SF, SF with the outer elements removed one by one, and full reciprocal processing (FRP) using the set of 15 unique TR pairs. Five different measurements were performed and almost identical values of SNR were obtained in each case (small error bars). The SNR decreased as the total number of TR pairs was reduced. Away from the geometric focus, the SNR of the SF cases greatly increased relative to the fixed-focus case.

The summary of SNR as a function of axial range is shown in FIG. 5 for the 38-MHz array and, like the beamwidth and DOF results, there was very little difference between the two array geometries. The SF cases were the same as described previously with the addition of the case representing FRP, FIG. 1(b). As would be expected, the full set of 25 TR pairs provided the best performance and SNR decreased as TR pairs were removed. This can be understood in terms of the magnitude of the peak signal decreasing as receive channels were removed (FIG. 4), whereas the RMS background noise increased as the total number of unique TR pairs decreased. The effect on background noise can be appreciated by comparing the full set of 25 TR pairs with the FRP case. Although the peak amplitude was the same for each case, the RMS noise was also coherently doubled for 10 of the TR pairs in the FRP case, which resulted in an SNR decrease of 2.5 dB. When the outer receive channels were removed one by one, the SNR decreased by 1.5, 2.5, 4.5, and 7.5 dB, respectively. As would be expected, the SNR of the fixed-focus case at the geometric focus was the same as full SF with 25 TR pairs and SNR dropped steeply when moving away from the geometric focus.

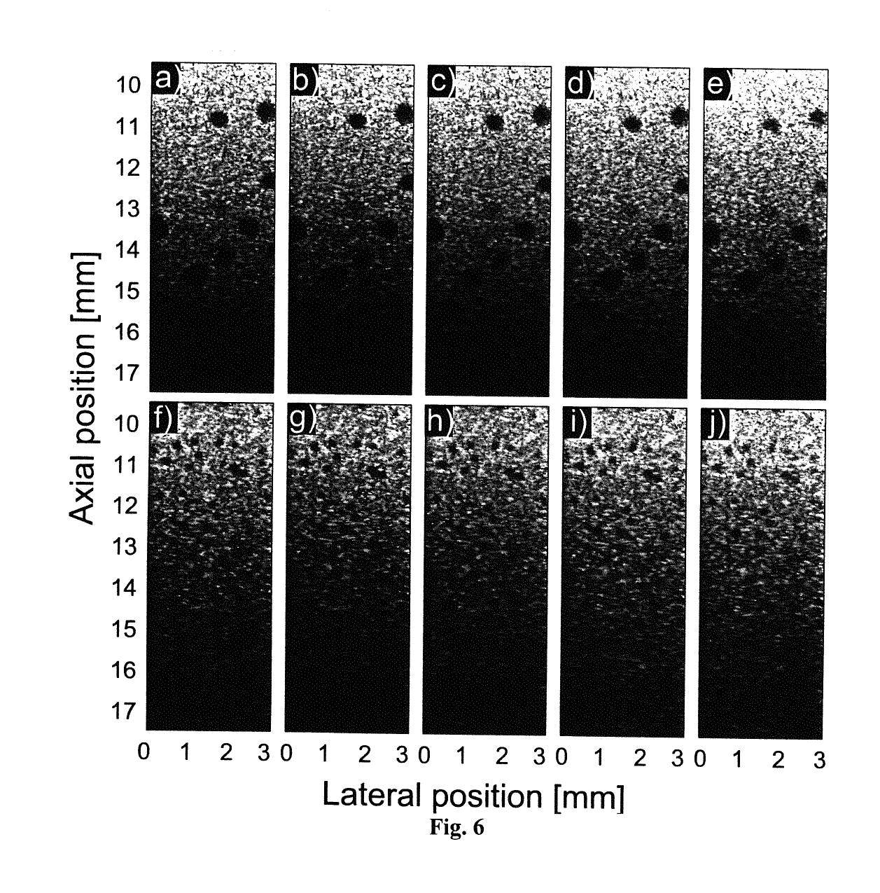

FIG. 6. shows images of 530—(a)-(e) and 200-μm (f)-(j) anechoic spheres obtained with the 38-MHz array when (a) and (f) are receiving on all elements and when removing (b) and (g) the outermost element, (c) and (h) outermost two, (d) and (i) outermost three, and (e) and (j) outermost four elements on receive. The transducer was located 9 mm above the surface of the phantom. All images are displayed with 80 dB of dynamic range and show the effect of reduced resolution, which resulted in higher noise levels in the spheres and degraded the definition and contrast of the image.

Image data were acquired with the 18- and 38-MHz arrays from all of the slabs of the anechoic-sphere phantom. FIG. 6 shows the B-mode images acquired in sections of the phantom embedded with 530—(FIG. 6(a)-6(e)) and 200-μm (FIG. 6(f)-6(j)) anechoic spheres. The image data were processed using the various SF strategies. When using all 25 TR pairs to form an SF image, the 530-μm anechoic spheres were visible to depths of 17 mm (FIG. 6(a)) as were the 200-μm (FIG. 6(f)). As the outer receive channels were removed one by one from the SF beamforming process (FIG. 6 from left to right), the resolution of the system was degraded (FIG. 3) which resulted in increased noise levels in the spheres. In the case of the 530-μm spheres, the main consequences were that the spheres appeared less contrasted with the background and the edges of the spheres had less definition. However, even with only one channel in receive, all the key features of the image were maintained (FIG. 6(e)). With the 200-μm spheres, which presented an inherently lower contrast because of their smaller size, the decrease in resolution further degraded the contrast of the spheres, and some of them could not be resolved by the system when using only one channel in receive (FIG. 6(j)). The SNR results obtained with the 18-MHz transducer when imaging the 1090- and 400-μm anechoic spheres showed similar trends as the spheres were reduced in size and fewer TR pairs were used for SF.

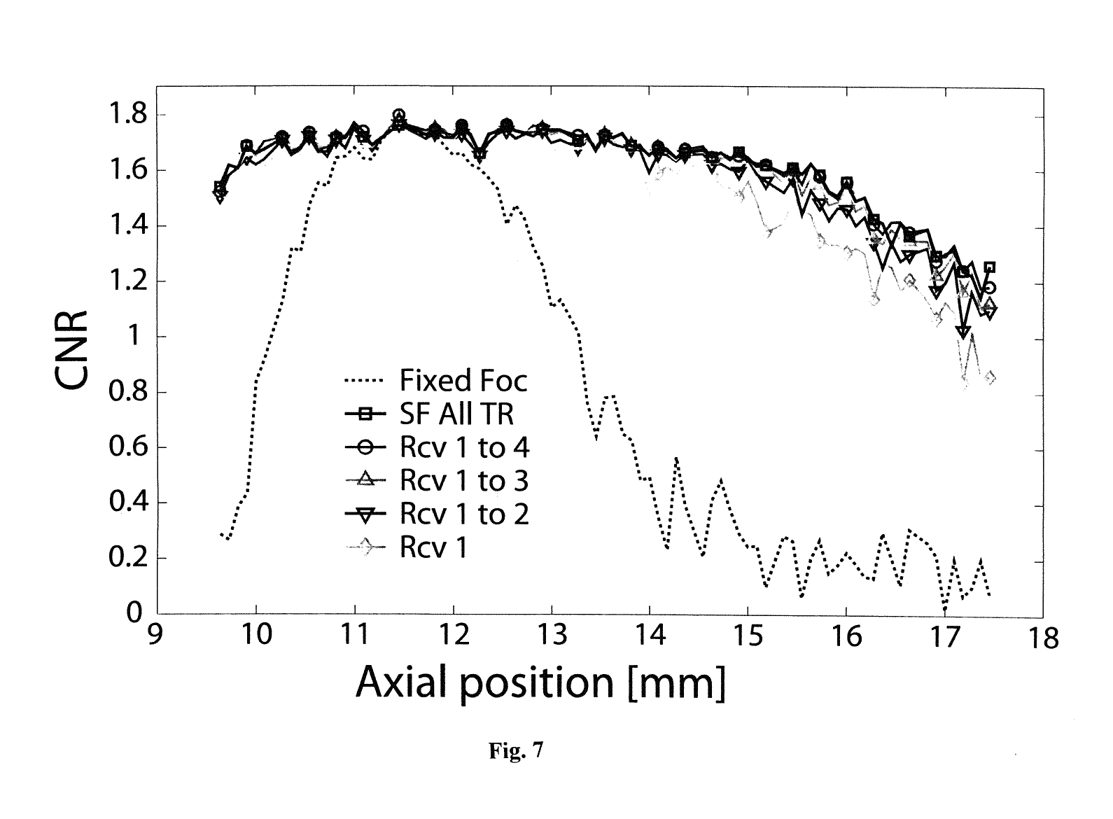

FIG. 7. shows CNRs of 530-μm anechoic spheres as a function of distance from the 38-MHz transducer, which was positioned 9 mm above the surface of the phantom. The CNR values of the spheres were obtained using fixed focusing or SF with the outer receive elements removed one by one. The CNR values were nearly identical for all SF cases, except for the deepest spheres when receiving with just the central element (diamond).

To better compare the imaging performances of the system when decreasing the number of receive channels, the CNR of the larger diameter spheres were calculated for the different SF approaches. The larger spheres were used because they could be detected with all SF strategies and the ROIs could be large enough that the characteristics of the envelope-detected RF signals could be more precise. The CNRs as a function of axial distance obtained with the 38-MHz array and the 530-μm anechoic spheres are plotted in FIG. 7 for the different SF approaches. The CNRs were nearly identical in the region of the geometric focus (12 mm) where SNR was at a maximum. Beyond the geometric focus, the CNRs slowly decreased with the steepest drop occurring for the SF case with just the central element receiving. For all of the SF approaches, CNRs remained at relatively high values of >1 (the theoretical maximum is 1.9) and provided a quantitative explanation as to why the anechoic spheres were well contrasted at all depths for all SF approaches (FIGS. 6(a)-6(e)). Similar results were obtained with the 18-MHz array when calculating the CNRs of the 1090-μm spheres.

Frame-rate benchmarks were obtained using a single pass approach with 251 scan lines, 50 μm between lines, 3500 RF points/line, 8-μs delay relative to the pulser trigger, and a 250-MHz sampling rate. The mechanical translation of the single pass took 150 ms. The software was split into three loops that passed data downstream from one loop to another via queue structures. Loop 1 consisted of the linear scan and transferring data to the host PC. Loop 2 received the data and performed SF along with subtraction of the mean from each RF line. Loop 3 flipped images taken in the reverse scan direction, applied a log-compression lookup table, and displayed the final B-mode image. It should be noted that absolute frame rates and the time spent in each loop are highly dependent on the motor, the properties of the PC motherboard, and the efficiency of the control software. Thus, the numbers reported should be interpreted relative to each other to show how the various SF strategies affect overall frame rate.

Table I shows the time spent in each loop per frame and the resulting frame rate for the various SF approaches. As receive channels were removed and fewer data were acquired, Loop 1 time decreased and began to approach the 150 ms that represented the actual mechanical scan time. The Loop 2 times also decreased because there were fewer data to process, but Loop 3 times remained fairly constant because the time was mostly devoted to the display of the image. In terms of frame rate, Loop 1 represented the limiting factor because Loops 2 and 3 operated downstream from Loop 1 and as long as their times were less than that of Loop 1, images did not stack up in the final display queue. The actual data throughput from the digitizer chassis to the host PC was difficult to determine from the Loop 1 times because the mechanical scan and data transfer times overlapped. The system software was modified to isolate the data transfer time from the mechanical motion time and then calculate a value of instantaneous data throughput of ≈72 MB/s (Table I).

TABLE I
SYNTHETIC-FOCUSING (SF) FRAME RATES
IN FRAMES PER SECOND (FPS)

| SF | Loop 1 | Loop 2 | Loop 3 | Frame rate | Burst throughput |
|---|---|---|---|---|---|
| method | (ms) | (ms) | (ms) | (fps) | (M13/s) |
| All 25 TR | 361 | 198 | 30 | 2.8 | 74 |
| Rev 1 to 4 | 300 | 158 | 30 | 3.3 | 76 |
| Rev I to 3 | 246 | 118 | 30 | 4.1 | 72 |
| Rev 1 to 2 | 194 | 80 | 30 | 5.2 | 70 |
| Rev 1 | 109 | 93 | 28 | 5.0 | 61 |

Transmit SF strategies provide a versatile means of gaining the full benefit of annular-array imaging without employing specialized TR focusing on all elements simultaneously. This approach to beamforming sacrifices overall signal strength because only a subset of the full transmit or full receive aperture is utilized, but the invention demonstrates that ultimate image quality is not compromised. The advantage to using this approach with an annular array is that the element count is low and it takes minimal time to acquire all TR data pairs at a single location. For a 38-MHz array that typically requires about 4 cm of roundtrip propagation, a single TR RF line can be acquired in 27 μs and the five transmit events needed to acquire all of the TR data pairs from a five-element annular array would take about 133 μs. Thus, for a fully optimized system with no limitations on motor speed, data transfer, or image processing, about 7500 image lines could be acquired in 1 s and an image with 300 lines could be sustained at 25 fps. A similar analysis for the 18-MHz array with an 8-cm round-trip distance yields a potential frame rate of 12 fps. If a single-transmit approach is used, the frame rates would increase by a factor of five to 125 fps for the 38-MHz transducer and 60 fps for the 18-MHz transducer. These frame rates are more than sufficient for the majority of ophthalmic and small-animal applications.

The preceding example assumes that the digitized data can be transferred and processed in real-time, a task that is not necessarily possible using CPU-based processors and the peripheral component interconnect (PCI) extended (PCI-X) to PCI-express (PCIe) bridge between the digitizer chassis and host PC. Data transfer via the PCI-X-to-PCIe bridge is limited to a maximum sustained bandwidth of 100 MB/s. In terms of digitizer memory, the benchmark scan parameters resulted in 22 MB of data for a full set of 25 TR pairs. Based on the results from Table I, an instantaneous throughput of ≈72 MB/s for full SF with 25 TR pairs was observed. However, the effective data throughput in terms of total data per second was 61 MB/s for the 25-TR-pairs case and then decreased as outer receive channels were removed. Thus, further improvements could be made in software to more efficiently handle data flow and reduce overhead. Using PCIe hardware would also improve bandwidth, resulting from potential data transfers of at least 250 MB/s per digitizer.

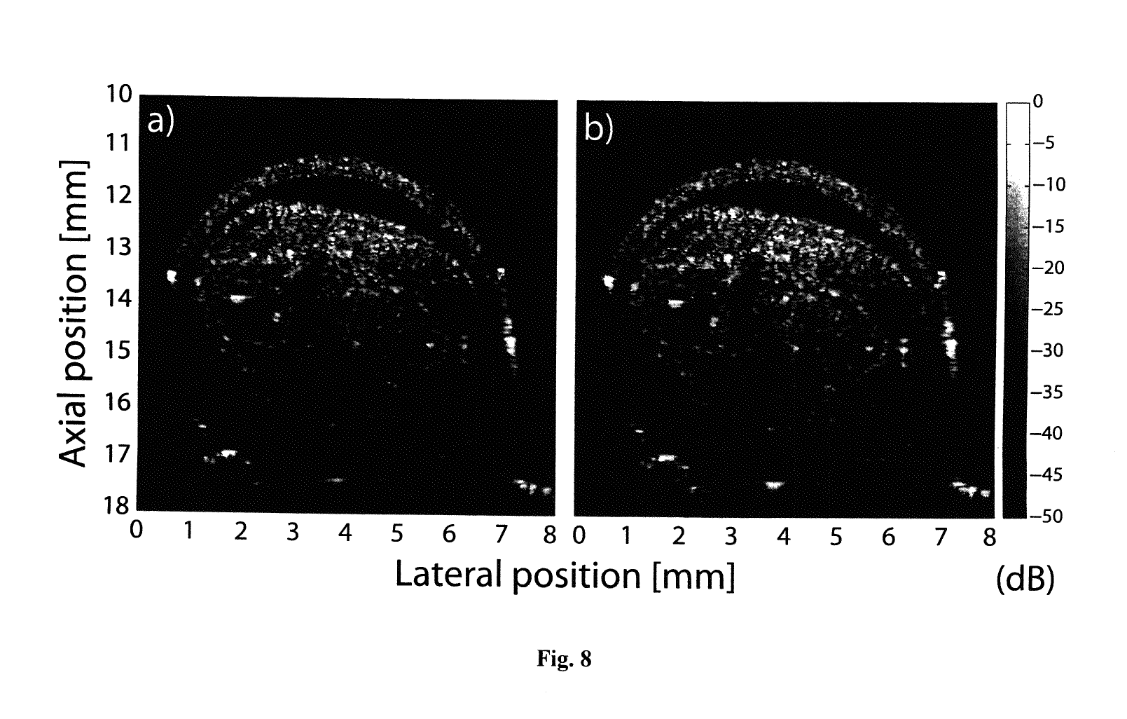

FIG. 8 shows B-mode images, using a 38-MHz annular array, of an externalized, in vivo mouse embryo 13 days after conception for (a) full SF with 25 TR pairs and (b) SF with the outer three receive elements removed. The SNR was 54 dB for the full SF case and 50 dB for the reduced SF case. Qualitatively, the two images are nearly identical; the full SF case has slightly less background noise and somewhat sharper definition for the edges.

In practice, the qualitative difference between SF approaches was relatively minor, as was seen with the anechoic-sphere phantom images (FIG. 6) and also with in vivo images of a mouse embryo (FIG. 8). These images were acquired from an externalized embryo. A case of full SF with 25 TR pairs (FIG. 8(a)) and an SF case with the outer three elements removed on receive (FIG. 8(b)) are shown. For a situation in which careful analysis of image data is necessary, such as brain ventricle segmentation, the full SF case will yield the most accurate results. For a situation in which the image is simply being used to locate and observe anatomical features, a partial SF case will be sufficient and will allow for the highest frame rate.

Although the beam properties were analyzed for only two specific annular arrays and a subset of all possible SF approaches, the results for the two array geometries showed nearly identical trends and can be used to draw some general conclusions about SF with annular arrays. First, DOF is maximized by using the central element on either transmit or receive because the central element has the broadest DOF. Once the central element is used, the overall amplitude of the DOF profile is dictated by how many of the TR pairs are used and can be understood in terms of how much of the full TR aperture is used. Second, lateral beamwidth is optimal when using all 25 TR pairs and removing outer TR pairs degrades resolution. Third, SNR also decreases as TR pairs are removed, with the full set of 25 TR pairs having the optimal SNR.

An alternate SF approach would be to remove the central element on receive (FIG. 1(d)). Because of reciprocal pairs, 24 TR pairs are effectively processed and the end results are very similar to the case of the outer receive element removed except that the −6-dB lateral beamwidth decreases slightly (≈2%) versus SF of the full set of 25 TR pairs. This arises because the central element contributes a wide lateral beamwidth to the overall acoustic field and its removal, even on just the receive side, lowers the overall lateral beamwidth. However, it is generally advantageous to make use of the central elements in transmit and receive because sensitivity typically decreases when moving toward the outer elements of an annular array.

SF strategies applied to an annular array permit a wide number of variations, from using the full TR aperture to various combinations of the total TR aperture. Once a full set of TR data are acquired, any one of the SF approaches can be applied to create an arbitrary number of focal zones Unlike SA approaches with a linear array, SF of an annular array only provides focusing along the acoustic axis and the annular array must be translated to form an image. Here, one subset of SF approaches was examined by comparing full SF with 25 TR pairs to SF cases in which the outer receive elements were removed, one by one, on receive but not on transmit.

Beam properties were presented for five-element annular arrays operating at 18 or 38 MHz with f-numbers of 3.1 and 2, respectively. The lateral beamwidth, DOF, CNR, and SNR trends as a function of axial range were seen to follow the same overall behavior for each array and the results can be extrapolated to general features of annular arrays. The invention shows that the optimal beam characteristics occurred when using all 25 TR pairs, as would be expected, and reducing the TR pairs used in processing degraded overall lateral beamwidth, lowered overall SNR, slightly lowered CNR, and lowered the overall DOF amplitude, but did not change the FWHM values of the DOF.

However, as the images of an anechoic-sphere phantom (FIG. 6) and an in vivo mouse embryo (FIG. 8) demonstrated, the SF images formed from a reduced set of TR pairs showed qualitative agreement with full SF of all TR pairs. Thus, the optimal SF approach depends on whether the final images must be acquired at a high frame rate or with a finer resolution.

The description of certain embodiments of this invention is intended to be illustrative and not limiting. Numerous other embodiments will be apparent to those skilled in the art, all of which are included within the broad scope of the invention. It is to be understood that the claims set forth herein cover all such alternative embodiments of the present invention.

1. The synthetic-focusing method of increasing the image formation speed in an annular array of N elements with a plurality of transmit and receive channels, the method comprising steps of;
providing excitation of the array elements in a predetermined sequence such that a round-trip acoustic path of consecutive elements does not overlap,
digitizing the acoustic echo received by the array elements with a digitizer for each channel;
selectively dropping a number of receive channels in order to achieve a reduction in the number of necessary digitizer channels and/or a reduction in the amount of data that is required to be digitized by the digitizers, or
selectively dropping a number of transmit channels to achieve a reduction in the number of necessary transmit channels and/or a reduction in the time required to obtain a set of image data and a reduction in the amount of data that is required to be digitized by the digitizers; and
processing the reduced amount of data digitized by the digitizer to form an image, the image being formed at a frame rate in excess of the frame rate that would have been achieved without the selective dropping of a number of receive or transmit channels.

2. The method in accordance with claim 1 wherein the dropping of transmit or receiving channels, drops either a transmit channel or a receive channel, but not both simultaneously.

3. The method in accordance with claim 2 wherein the amplitude of an acoustic signal present on a first pair of transmit and receive channels is doubled if said first pair of transmit and receive channels is the reciprocal of a second pair of transmit and receive channels and the second pair of transmit and receive channels are not used to form the image.

4. The method in accordance with claim 1 wherein said transmit and receive channels in said annular array include outer transmit and receive channels and central transmit and receive channels.

5. The method in accordance with claim 4 wherein lateral resolution of said image degrades as transmit or receive channels are selectively dropped.

6. The method in accordance with claim 5 wherein dropping one outermost receive channel degrades lateral resolution by a first percentage and dropping an increasing number of outer receive channels degrades lateral resolution by a second percentage, said second percentage being greater than said first percentage.

7. The method in accordance with claim 6 wherein dropping the outer receive channels progressively lowers an amplitude profile of a depth of field parameter for said ultrasound system.

8. The method in accordance with claim 1 wherein said digitized data can be processed in real-time.

9. The method in accordance with claim 1 wherein selectively dropping transmit and receive channels increases frame rate without significant degradation of said framed image.

10. The method in accordance with claim 9 wherein a change in lateral resolution of said framed image is a function of the total number of transmit or receive channels dropped.

11. The method in accordance with claim 6 wherein dropping the central receive channels progressively lowers an amplitude profile of a depth of field parameter for said ultrasound system.

12. The method in accordance with claim 1 wherein only a single transmit channel is used and one or more receive channels are used.
